(19) United States
(12) Patent Application Publication
(10) Pub. No.: US 2017/0325905 A1
(43) Pub. Date: Nov. 16, 2017

(54) MEDICAL MANIPULATOR

(71) Applicant: OLYMPUS CORPORATION, Tokyo (JP)

(72) Inventors: Shuya JOGASAKI, Tokyo (JP); Masatoshi IIDA, Tokyo (JP); Noriaki YAMANAKA, Tokyo (JP); Mitsuhiro HARA, Tokyo (JP)

(73) Assignee: OLYMPUS CORPORATION, Tokyo (JP)

(21) Appl. No.: 15/665,462

(22) Filed: Aug. 1, 2017

Related U.S. Application Data
(63) Continuation of application No. PCT/JP2015/061903, filed on Apr. 17, 2015.

(51) Int. Cl. A61B 34/00; B25J 9/10; B25J 18/06

(52) U.S. Cl. CPC A61B 34/74; B25J 18/06; B25J 9/102; B25J 9/1045

(57) ABSTRACT

A medical manipulator including: a rotating joint that rotates an end effector disposed at a distal end thereof about a first axis; a flexing joint that is disposed on a base-end side of the rotating joint and that pivots the end effector about a second axis that intersects the first axis; a drive portion that generates a rotational driving force; a motive-power transmitting member that transmits the rotational driving force generated by the drive portion to the rotating joint by passing through the flexing joint; and a speed-reduction portion that transmits the rotational driving force transmitted thereto by the motive-power transmitting member to the end effector after performing speed-reduction.

### CROSS-REFERENCE TO RELATED APPLICATIONS

This is a continuation of International Application PCT/JP2015/061903 which is hereby incorporated by reference herein in its entirety.

### TECHNICAL FIELD

The present invention relates to a medical manipulator.

### BACKGROUND ART

There are known surgical treatment tools that have a rotating joint at a distal end of a flexing joint and that rotate a gripping portion supported by the rotating joint about the axis thereof (for example, see Patent Literatures 1 and 2).

With these surgical treatment tools, the attitude of the gripping portion is changed by actuating the flexing joint, a gripping target (suturing needle or the like) is clamped by the gripping portion, after which the gripping portion is rotated by actuating the rotating joint, and thus, the gripped gripping target is rotated.

### CITATION LIST

### Patent Literature

{PTL 1} Publication of U.S. Pat. No. 6,746,443
{PTL 2} Publication of U.S. Pat. No. 6,676,684

### SUMMARY OF INVENTION

An aspect of the present invention is a medical manipulator including: a rotating joint that rotates an end effector disposed at a distal end thereof about a first axis; a flexing joint that is disposed on a base-end side of the rotating joint and that pivots the end effector about a second axis that intersects the first axis; a drive portion that generates a rotational driving force; a motive-power transmitting member that transmits the rotational driving force generated by the drive portion to the rotating joint by passing through the flexing joint; and a speed-reduction portion that transmits the rotational driving force transmitted thereto by the motive-power transmitting member to the end effector after performing speed-reduction.

### BRIEF DESCRIPTION OF DRAWINGS

FIG. 1 is an overall configuration diagram showing a medical manipulator system provided with a medical manipulator according to an embodiment of the present invention.

FIG. 2 is a perspective view showing the medical manipulator in FIG. 1.

FIG. 3 is a longitudinal cross-sectional view showing a distal-end portion of the medical manipulator in FIG. 2.

FIG. 4 is a front view showing a speed-reduction portion of the medical manipulator in FIG. 2.

FIG. 5 is a longitudinal cross-sectional view showing a state in which a flexing joint of the medical manipulator in FIG. 3 is flexed.

FIG. 6 is a perspective view showing a modification of the speed-reduction portion in FIG. 4.

FIG. 7 is a longitudinal cross-sectional view of a distal-end portion showing a modification of the medical manipulator in FIG. 2.

FIG. 8 is a longitudinal sectional view of the distal-end portion of the medical manipulator in FIG. 7, taken through another sectional plane.

### DESCRIPTION OF EMBODIMENT

A medical manipulator 3 according to an embodiment of the present invention will be described below with reference to the drawings.

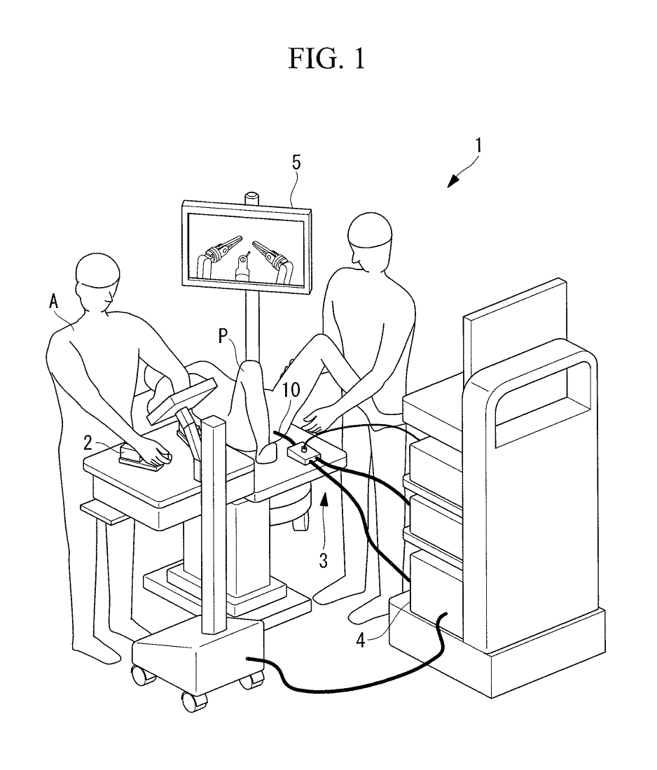

The medical manipulator 3 according to this embodiment is employed in, for example, a medical manipulator system 1 shown in FIG. 1. This medical manipulator system 1 is provided with: a manipulation input device 2 that is manipulated by an operator A; the medical manipulator 3, which is inserted into a body cavity of a patient P; a control portion 4 that controls the medical manipulator 3 on the basis of manipulations input via the manipulation input device 2; and a monitor 5.

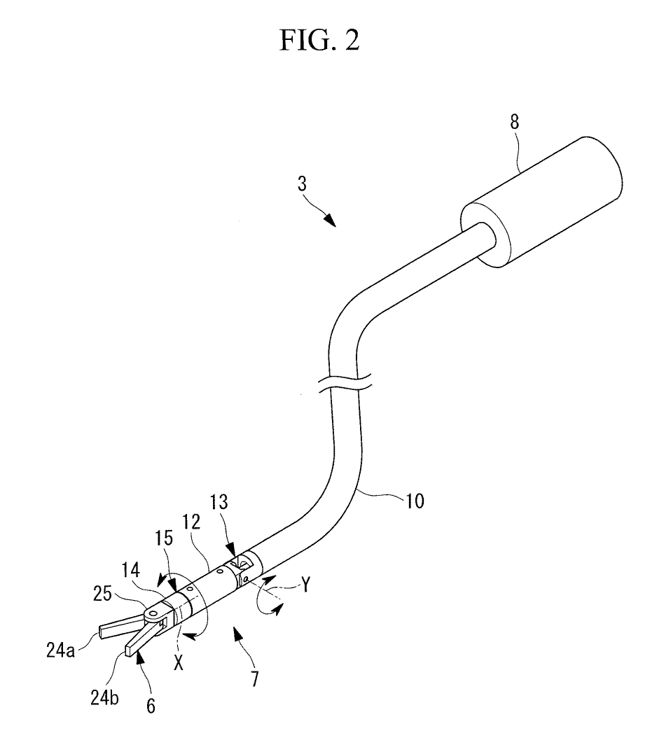

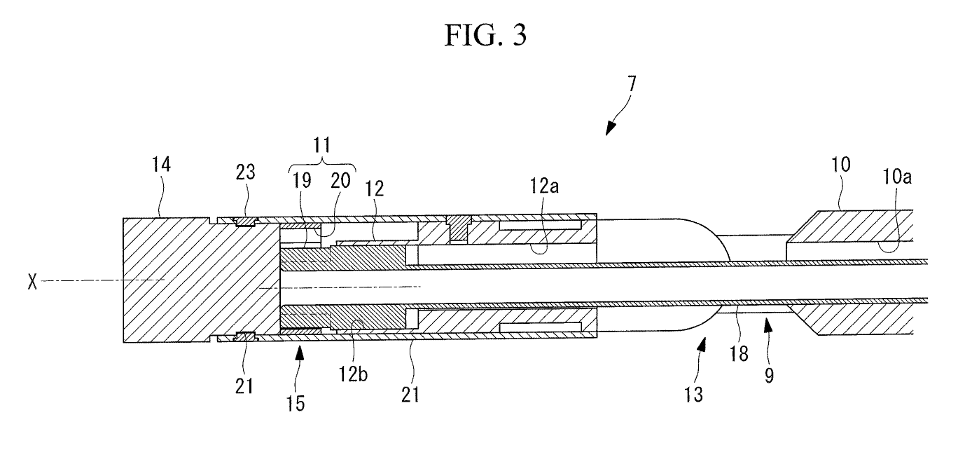

As shown in FIGS. 2 and 3, the medical manipulator 3 according to this embodiment is provided with, for example: an elongated inserted portion 10 that is inserted into the body cavity of the patient P via a channel in an endoscope that is inserted into the body cavity of the patient P; a movable portion 7 that is disposed at a distal end of the inserted portion 10 and that supports an end effector 6 at a distal end thereof; a rotational drive portion (drive portion) 8 that is disposed at a base end of the inserted portion 10 and that actuates the movable portion 7 by being controlled by the control portion 4; a motive-power transmitting member 9 that transmits the rotational driving force generated by the rotational drive portion 8 to the movable portion 7; and a speed-reduction portion 11 that transmits the rotational driving force transmitted thereto by the motive-power transmitting member 9 to the movable portion 7 after speed-reduction.

The movable portion 7 is provided with a flexing joint 13 that pivots a first joint member 12 about an axis (second axis) Y that is orthogonal to the longitudinal axis of the inserted portion 10 and a rotating joint 15 that is disposed at a distal end of the first joint member 12 of the flexing joint 13 and that rotates a second joint member 14 about an axis (first axis) X that is orthogonal to the second axis Y. The second joint member 14 is secured to the end effector 6.

By pivoting the first joint member 12 by actuating the flexing joint 13, it is possible to pivot the rotating joint 15, which is disposed at the distal end of the first joint member 12, and the end effector 6 about the second axis Y. Although the rotational driving force can be transmitted from the rotational drive portion 8 to the flexing joint 13 by means of, for example, pulleys 16 and wires 17, descriptions thereof will be omitted here.

In addition, by rotating the second joint member 14 by actuating the rotating joint 15, it is possible to rotate the end effector 6 about the first axis X. As shown in FIG. 3, the motive-power transmitting member 9 that transmits the rotational driving force from the rotational drive portion 8 to the rotating joint 15 is constituted of a torque tube 18.

The inserted portion 10 and the first joint member 12 are both configured to be hollow, and, as shown in FIG. 3, the torque tube 18 is disposed so as to pass through the hollow flexing joint 13 and so as to extend from the inserted portion 10 to the vicinity of the second joint member 14.

The torque tube 18 is a hollow tube possessing flexibility and is configured so that a wire (not shown) can be made to pass through the interior thereof. The wire is used to actuate the end effector 6 secured to the second joint member 14.

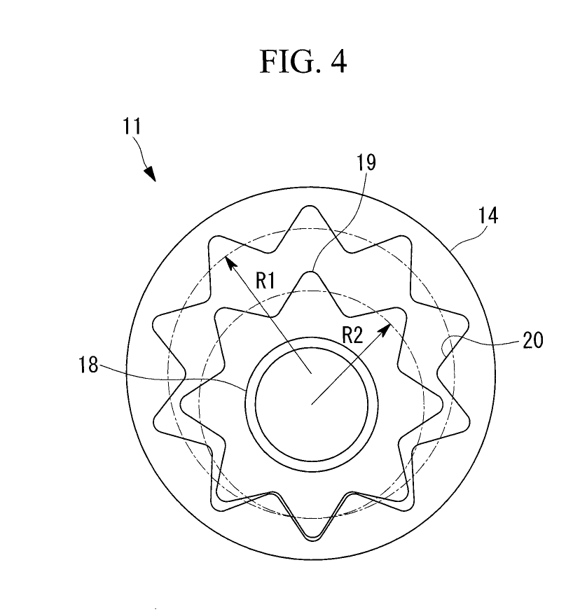

The speed-reduction portion 11 is provided with an input-side gear 19 that is secured to a distal end of the torque tube 18 and an output-side gear 20 that is secured to the second joint member 14. As shown in FIG. 4, the input-side gear 19 is, for example, a gear that has external teeth. In addition, the output-side gear 20 is, for example, a gear that is formed in a cylindrical inner surface and that has internal teeth. The input-side gear 19 and the output-side gear 20 are eccentrically disposed so as to have the rotation axes thereof parallel to each other and so as to be engaged with each other. The number of teeth on the input-side gear 19 (for example, eight) is less than the number of teeth on the output-side gear 20 (for example, ten). Accordingly, the rotational speed of the output-side gear 20 is decreased with respect to the rotational speed of the input-side gear 19 by a speed-reduction ratio determined by the ratio of the numbers of the teeth.

In other words, as shown in FIG. 4, a pitch circle radius R1 of the output-side gear 20, which is formed in the cylindrical inner surface, is greater than a pitch circle radius R2 of the input-side gear 19, and the input-side gear 19 is supported by means of a fitting hole 12b formed in the first joint member 12 so as to be eccentrically disposed in one radial direction with respect to the output-side gear 20 and so as to be constantly engaged therewith.

The second joint member 14 is fitted, in a rotatable manner, in a cylindrical cover member 21 secured to the first joint member 12. A circumferential groove 22 is formed in the second joint member 14, and the second joint member 14 is held by a pin 23 that passes through the cover member 21 in the radial direction, that is secured thereto, and that protrudes into the circumferential groove 22, so as not to fall out in the direction of the first axis X.

The operation of the thus-configured medical manipulator 3 according to this embodiment will be described below.

In order to treat an affected portion in the body by using the medical manipulator system 1 according to this embodiment, the medical manipulator 3 according to this embodiment is inserted, from the end effector 6 at the distal end thereof, via the channel in the inserted portion of the endoscope inserted into the body cavity from outside the body of the patient P, and the end effector 6 and the movable portion 7 are made to protrude from an opening of the channel at the distal-end surface of the inserted portion of the endoscope disposed in the body.

When the operator A performs manipulation inputs by manipulating the manipulation input device 2 in the state in which the end effector 6 and the movable portion 7 are protruded, the control portion 4 controls the rotational drive portion 8 in accordance with the manipulation inputs, thus actuating the movable portion 7. By actuating the flexing joint 13, the end effector 6 is pivoted about the second axis Y, and thus, the attitude thereof is changed. In addition, by actuating the rotating joint 15, the end effector 6 is rotated about the first axis X.

In the case in which the end effector 6 has a gripping portion 25 that can open/close a pair of gripping pieces 24a and 24b, it is possible to change the opening/closing directions of the gripping pieces 24a and 24b by actuating the rotating joint 15.

In this case, with the medical manipulator 3 according to this embodiment, the rotational driving force generated by the rotational drive portion 8 is transmitted by the torque tube 18, which is disposed by passing through hollow portions 10a and 12a provided in the inserted portion 10 and the first joint member 12, thus rotating the input-side gear 19, which is engaged with the output-side gear 20 secured to the second joint member 14, which is secured to the end effector 6. Because the number of teeth on the input-side gear 19 is less than the number of teeth on the output-side gear 20, the rotational driving force transmitted by the torque tube 18 is amplified in accordance with the speed-reduction ratio when being transmitted to the output-side gear 20, and thus, the end effector 6 is rotated.

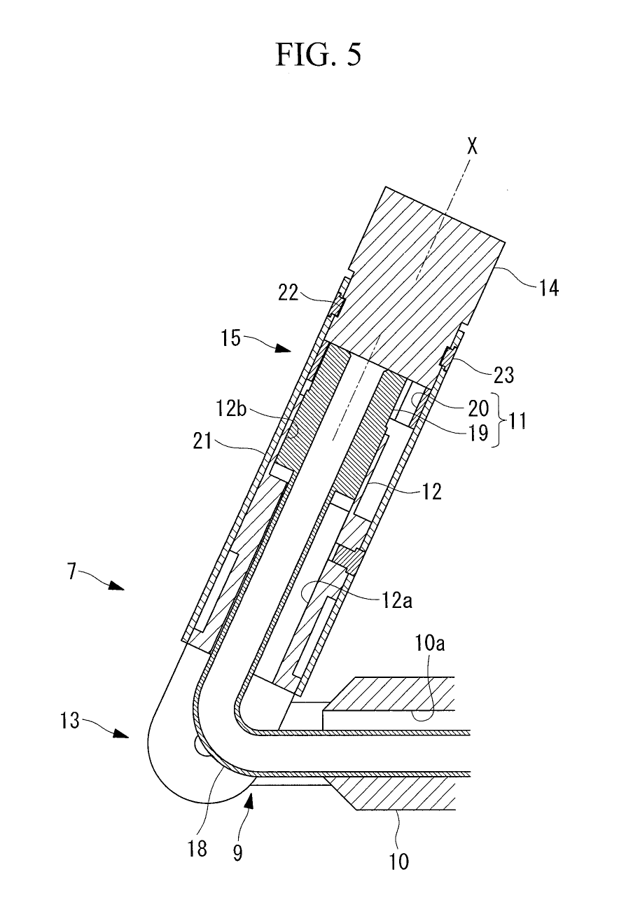

In other words, as shown in FIG. 5, when the attitude of the end effector 6 is changed by flexing the flexing joint 13 of the medical manipulator 3, the torque tube 18 passing through the flexing joint 13 is also flexed and is brought into close contact with inner surfaces of the hollow portions 10a and 12a of the inserted portion 10 and the first joint member 12. As a result, when rotating the torque tube 18, the friction between the torque tube 18 and the members in the surrounding areas thereof is increased, and thus, the efficiency at which the rotational driving force is transmitted is decreased.

With the medical manipulator 3 according to this embodiment, because the speed-reduction portion 11 is configured on the basis of the difference between the number of teeth on the input-side gear 19, which is secured to the distal end of the torque tube 18, and that of the output-side gear 20, which is secured to the end effector 6, there is an advantage in that it is possible to restore the rotational driving force in the speed-reduction portion 11 even if the transmission efficiency is decreased in the torque tube 18, and thus it is possible to more reliably rotate the end effector 6.

In addition, with the medical manipulator 3 according to this embodiment, because the output-side gear 20 has the internal teeth formed on the cylindrical inner surface, it is possible to dispose the input-side gear 19 in a state in which the input-side gear 19 is engaged with the output-side gear 20 on the inner side thereof. As a result, there is an advantage in that it is possible to reduce the diameter of the speed-reduction portion 11.

Note that, in this embodiment, although the output-side gear 20 is configured with the cylindrical gear having the internal teeth, in the case in which there is an allowance in the external diameter, a gear having external teeth on the outer surface of a cylinder may be employed as the output-side gear 20.

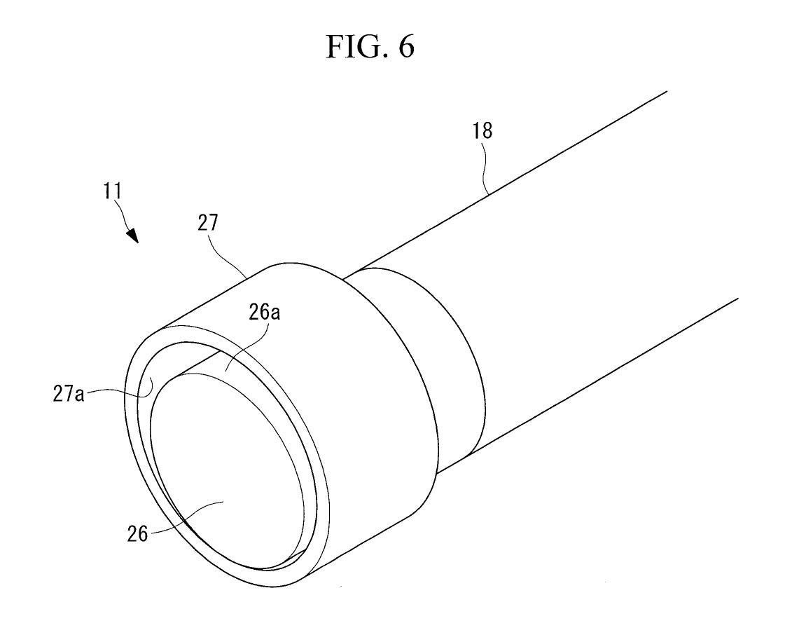

In addition, although the rotational driving force is transmitted by means of engagement between the input-side gear 19 and the output-side gear 20, alternatively, as shown in FIG. 6, it is permissible to employ a component having a structure in which the rotational driving force is transmitted by means of friction by bringing a friction surface 26a disposed on an outer circumference of a columnar input-side rotator 26 into contact with a friction surface 27a disposed on an inner circumference of a cylindrical output-side rotator 27.

In this case also, by setting the diameter at the friction surface 27a of the output-side rotator 27 so as to be greater than the diameter at the friction surface 26a of the input-side rotator 26, speed-reduction occurs when transmitting the rotational driving force, as with the case in which gears are employed, and thus, it is possible to amplify the rotational driving force.

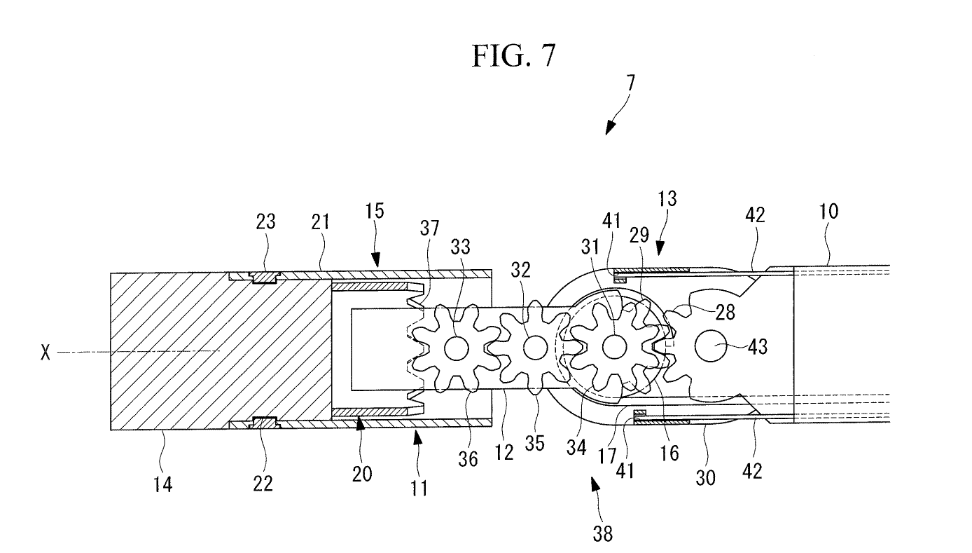

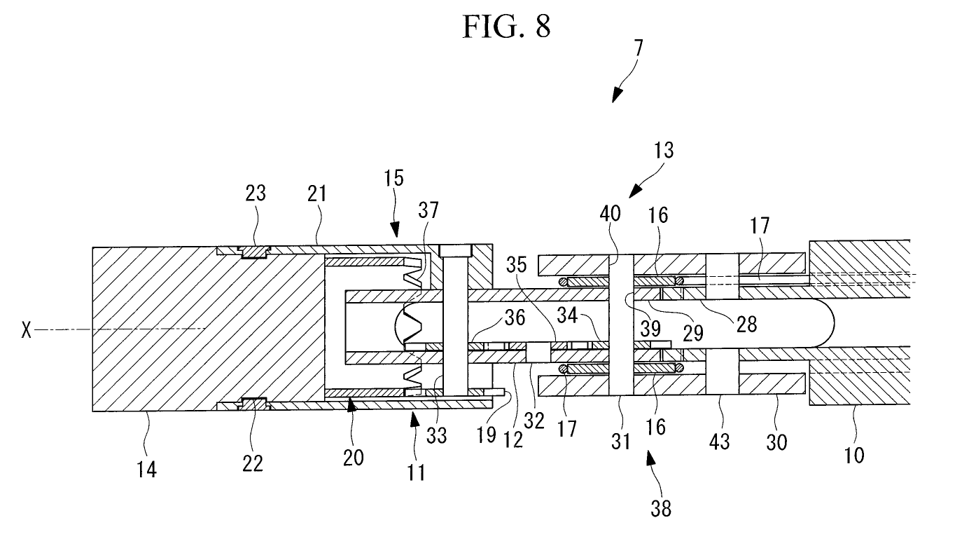

In addition, in this embodiment, although the rotational driving force generated by the rotational drive portion 8 is transmitted by means of the torque tube 18, alternatively, as shown in FIGS. 7 and 8, the rotational driving force may be transmitted by means of the wires 17. In FIGS. 7 and 8, a so-called double joint system is employed as the flexing joint 13, which is configured by engaging spur gears 28 and 29 secured to the end portions of the inserted portion 10 and the first joint member 12 with each other and by linking the spur gears 28 and 29 by means of a linkage member 30 between the two axes of the respective spur gears 28 and 29, said axes being parallel to each other.

As shown in FIG. 8, with this medical manipulator 3, three shafts 31, 32, and 33 that are parallel to the axis Y of the flexing joint 13 are provided in the first joint member 12 of the rotating joint 15, which is disposed further forward than the flexing joint 13 is. The first shaft 31, which is closest to the base-end side, is disposed along a distal-end-side axis of the flexing joint 13 and is cut so as to take a D-shape. The two pulleys 16 and a first spur gear 34 are secured to this first shaft 31 so that the D-cut allows rotation thereof as a single unit. In addition, the spur gear 29 and through-holes 39 and 40 of the linkage member 30 are not cut so as to take a D-shape, and thus, the first shaft 31 can be rotated relative to the spur gear 29 and the linkage member 30.

The wires 17 are individually threaded around the two pulleys 16 in opposite directions, and end portions of the individual wires 17 are secured to the pulleys 16. By doing so, by pulling one of the wires 17 toward the base end, the corresponding pulley 16 is rotated in the opposite direction, and thus, it is possible to rotate the first spur gear 34 in one of the two directions via the first shaft 31.

The second spur gear 35 that engages with the first spur gear 34 is secured to the second shaft 32 that is adjacent to the first shaft 31. The third spur gear 36, which engages with the second spur gear 35, and the input-side gear 19, which is formed of spur gears forming the speed-reduction portion 11, are secured to the third shaft 33, which is adjacent to the second shaft 32.

The cylindrical output-side gear 20, which is provided with a face gear 37 that engages with the input-side gear 19, is secured to the second joint member 14 of the rotating joint 15.

As shown in FIG. 7, the linkage member 30 is provided, on both sides in the pivoting direction of the linkage member 30, with securing portions 41 to which wires 42, which are separate from the wires 17, are secured. By pulling one of the wires 42 toward the base end, the linkage member 30 is pivoted about a shaft 43 with respect to the inserted portion 10, and, by doing so, it is possible to flex the flexing joint 13 by pivoting the first joint member 12 about the first shaft 31 with respect to the linkage member 30.

In other words, in this example, the wires 17, the pulleys 16, and the first spur gear 34 to the third spur gear 36 constitute a motive-power transmitting member 38 that transmits the rotational driving force generated by the rotational drive portion 8, and the input-side gear 19 is rotationally driven by the rotational driving force transmitted by the motive-power transmitting member 38.

In the above-described embodiment, although the speed-reduction portion 11 transmits the rotational driving force while performing speed-reduction between the gears 19 and 20, which are rotated about axes that are parallel to each other, in examples in FIGS. 7 and 8, the rotational driving force is transmitted while performing speed-reduction between the spur gear 19 and the face gear 37, which are rotated about axes that are orthogonal to each other.

By doing so, although it is also possible to prevent, by means of flexing of the flexing joint 13, the rotational driving force for driving the rotating joint 15 from being decreased due to friction, transmission by the plurality of gears 34, 35, and 36 causes a decrease in the transmission efficiency. Therefore, by restoring the rotational driving force by using the speed-reduction portion 11, it is possible to more reliably rotate the end effector 6. The second spur gear 35, which is an intermediate gear, may be omitted.

Note that the method for transmitting the motive power between the axes that are orthogonal to each other is not limited to the combination of the spur gear 19 and the face gear 37, and a combination of a helical gear and the face gear 37, a combination of helical gears with each other, and so forth may be employed.

The above-described embodiment leads to the following invention.

An aspect of the present invention is a medical manipulator including: a rotating joint that rotates an end effector disposed at a distal end thereof about a first axis; a flexing joint that is disposed on a base-end side of the rotating joint and that pivots the end effector about a second axis that intersects the first axis; a drive portion that generates a rotational driving force; a motive-power transmitting member that transmits the rotational driving force generated by the drive portion to the rotating joint by passing through the flexing joint; and a speed-reduction portion that transmits the rotational driving force transmitted thereto by the motive-power transmitting member to the end effector after performing speed-reduction.

With this aspect, when the rotational driving force is generated by actuating the drive portion, the generated rotational driving force is transmitted to the rotating joint, which is located further forward, by the motive-power transmitting member by passing through the flexing joint. Then the rotational driving force transmitted to the rotating joint is transmitted to the end effector in the state in which speed-reduction is applied by the speed-reduction portion. In other words, although, when the flexing joint is flexed, the motive-power transmitting member passing through the flexing joint comes into contact with the members in the surrounding areas, thus generating friction, because the speed-reduction portion after passing through the flexing joint amplifies the rotational driving force, the rotational driving force that has been lost due to the friction is restored by the speed-reduction portion, and thus, it is possible to drive the rotating joint by using a high torque.

With the above-described aspect, the speed-reduction portion may be provided with an input-side gear provided in the motive-power transmitting member and an output-side gear that is secured to the end effector so as to be rotatable about the first axis and that engages with the input-side gear, and number of teeth on the output-side gear may be greater than number of teeth on the input-side gear.

By doing so, the speed-reduction ratio is determined by the ratio of the number of teeth on the input-side gear and the number of teeth on the output-side gear, and the rotational driving force transmitted by the input-side gear is amplified by the engagement with the output-side gear, and thus, it is possible to easily rotate the end effector secured to the output-side gear about the first axis.

In addition, with the above-described aspect, the input-side gear may be provided so as to be rotatable about an axis that is parallel to the first axis.

By doing so, it is possible to transmit the rotational driving force transmitted by the motive-power transmitting member from the input-side gear to the output-side gear that are rotated about axes that are parallel to each other. Because conversion in the direction of rotation is not associated with this process, it is possible to amplify the rotational driving force by performing speed-reduction on the rotational speed by using a relatively small diameter.

In addition, with the above-described aspect, the motive-power transmitting member may be provided with a wire that transmits a tensile force by being pulled by the drive portion and a pulley that is provided so as to be rotatable about an axis that is parallel to the second axis by the tensile force of the wire, and the input-side gear may be rotated about an axis that is parallel to the second axis by the rotation of the pulley.

By doing so, the rotational driving force of the drive portion transmitted by the wire rotates the pulley about the axis that is parallel to the second axis, and the rotation of the pulley causes the input-side gear to be rotated about the axis that is parallel to the second axis. Because the input-side gear is rotated about the axis that is parallel to the second axis about which the flexing joint is rotated, it is possible to suppress a decrease in the efficiency at which the rotational driving force is transmitted due to flexing of the flexing joint.

In addition, with the above-described aspect, the motive-power transmitting member may be formed of a torque tube that possesses flexibility and that is disposed so as to pass through the flexing joint, and the input-side gear may be secured to a distal end of the torque tube.

By doing so, although the friction between the torque tube and the members in the surrounding areas is increased when the torque tube having flexibility is bent by flexing of the flexing joint, thus decreasing the efficiency at which the rotational driving force is transmitted, because the speed-reduction portion provided with the input-side gear secured to the distal end of the torque tube and the output-side gear increases the rotational driving force, the rotational driving force that has been lost due to the friction is restored by the speed-reduction portion, and thus, it is possible to drive the rotating joint by using a high torque.

In addition, with the above-described aspect, the speed-reduction portion may be provided with an input-side rotator that is provided in the motive-power transmitting member and an output-side rotator that is secured to the end effector and that transmits the rotational driving force between the output-side rotator and the input-side rotator by means of friction, and a diameter of a friction surface of the output-side rotator may be greater than a diameter of a friction surface of the input-side rotator.

By doing so, the speed-reduction ratio is determined by the ratio of the diameter of the friction surface of the input-side rotator and the diameter of the friction surface of the output-side rotator, and the rotational driving force transmitted, by means of friction, to the input-side rotator is amplified when being transmitted to the output-side rotator, and thus, it is possible to easily rotate the end effector secured to the output-side rotator about the first axis.

### REFERENCE SIGNS LIST

3 medical manipulator
6 end effector
8 rotational drive portion (drive portion)
9, 38 motive-power transmitting member
11 speed-reduction portion
13 flexing joint
15 rotating joint
16 pulley
17 wire
18 torque tube
19 input-side gear
20 output-side gear
26 input-side rotator
26a, 27a friction surface
27 output-side rotator
X first axis
Y second axis

1. A medical manipulator comprising:
a rotating joint that rotates an end effector disposed at a distal end thereof about a first axis;
a flexing joint that is disposed on a base-end side of the rotating joint and that pivots the end effector about a second axis that intersects the first axis;
a drive portion that generates a rotational driving force;
a motive-power transmitting member that transmits the rotational driving force generated by the drive portion to the rotating joint by passing through the flexing joint; and
a speed-reduction portion that transmits the rotational driving force transmitted thereto by the motive-power transmitting member to the end effector after performing speed-reduction.

2. A medical manipulator according to claim 1,
wherein the speed-reduction portion is provided with an input-side gear provided in the motive-power transmitting member and an output-side gear that is secured to the end effector so as to be rotatable about the first axis and that engages with the input-side gear, and
number of teeth on the output-side gear is greater than number of teeth on the input-side gear.

3. A medical manipulator according to claim 2, wherein the input-side gear is provided so as to be rotatable about an axis that is parallel to the first axis.

4. A medical manipulator according to claim 2,
wherein the motive-power transmitting member is provided with a wire that transmits a tensile force by being pulled by the drive portion and a pulley that is provided so as to be rotatable about an axis that is parallel to the second axis by the tensile force of the wire, and
the input-side gear is rotated about an axis that is parallel to the second axis by the rotation of the pulley.

5. A medical manipulator according to claim 2,
wherein the motive-power transmitting member is formed of a torque tube that possesses flexibility and that is disposed so as to pass through the flexing joint, and
the input-side gear is secured to a distal end of the torque tube.

6. A medical manipulator according to claim 1,
wherein the speed-reduction portion is provided with an input-side rotator that is provided in the motive-power transmitting member and an output-side rotator that is secured to the end effector and that transmits the rotational driving force between the output-side rotator and the input-side rotator by means of friction, and
a diameter of a friction surface of the output-side rotator is greater than a diameter of a friction surface of the input-side rotator.
